(19) United States
(12) Patent Application Publication
(10) Pub. No.: US 2004/0224364 A1
(43) Pub. Date: Nov. 11, 2004

(54) Organo-zinc compounds

(75) Inventor: Reuben D. Rieke, Lincoln, NE (US)

Correspondence Address: Merchant & Gould P.C., P.O. Box 2903, Minneapolis, MN, 55402-0903, US

(73) Assignee: Rieke Metals, INc.

(21) Appl. No.: 10/841,790

(22) Filed: May 7, 2004

Related U.S. Application Data
(60) Provisional application No. 60/469,520, filed on May 9, 2003.

(51) Int. Cl. C07F 3/06; G01N 33/53

(52) U.S. Cl. 435/7.1; 514/184; 544/64; 546/2; 548/102; 548/402

(57) ABSTRACT

The invention includes compounds of formula I:

X—A—Y—Z (I)

wherein X is —ZnBr, —ZnCl, or —ZnI; A is an aryl, or heteroaryl; Y is C₁-C₆ alkyl; and Z is a N containing heteroaryl that is bonded by the N. The invention also includes combinatorial libraries made using at least one compound of the invention.

This application claims priority of invention under 35 U.S.C. §119(e) from U.S. provisional application No. 60/469,520, filed on May 9, 2003, entitled ORGANO-ZINC COMPOUNDS, the disclosure of which is incorporated by reference herein.

### BACKGROUND

Combinatorial chemistry is becoming a very important tool in creating large collections of small organic compounds or libraries that can be screened for pharmacological or biological activity. The reactions that create the libraries are generally performed in solution or in the solid phase. Typically, combinatorial methods involve the addition of various structural components either in a controlled or random manner to a core chemical structure in order to produce all or a substantial portion of the possible combinations that can result from the different possible reactions at each stage.

Therefore there remains a need for efficient methods for obtaining such libraries of compounds and the compounds making up those libraries.

### SUMMARY OF THE INVENTION

The invention includes compounds of formula I:

X—A—Y—Z (I)

wherein X is —ZnCl, —ZnBr, or —ZnI;

A is an aryl, or heteroaryl;

Y is C₁-C₆ alkyl; and

Z is a N containing heteroaryl that is bonded by the N.

The invention also includes combinatorial libraries produced using compounds of formula I.

### DESCRIPTION OF THE INVENTION

The recitation of numerical ranges by endpoints includes all numbers and fractions subsumed within that range (e.g. 1 to 5 includes 1, 1.5, 2, 2.75, 3, 3.80, 4, and 5).

All numbers and fractions thereof are presumed to be modified by the term “about.”

It is to be understood that “a,” “an,” and “the” include plural referents unless the content clearly dictates otherwise. Thus, for example, reference to a composition containing “a compound” includes a mixture of two or more compounds.

Compounds of the Invention

The invention includes halogen substituted amines, and organometallic reagents that can be prepared therefrom. Examples of organometallic reagents that are included in the invention include, but are not limited to, Grignard reagents, lithium reagents, potassium reagents, sodium reagents, zinc reagents, mercury reagents, lead reagents, and thallium reagents formed from the halogen substituted amines. Compounds in which other metal atoms are bonded to a carbon atom of the halogen substituted amine are also included in the invention. Compounds of the invention include compounds of formula I below.

X—A—Y—Z (I)

wherein X is —ZnCl, —ZnBr, or —ZnI;

A is an aryl, or heteroaryl;

Y is C₁-C₆ alkyl; and

Z is a N containing heteroaryl that is bonded by the N.

As used herein the term “aryl” refers to unsaturated aromatic carbocyclic radicals having a single ring, such as phenyl, or multiple condensed rings, such as naphthyl or anthryl. The term “aryl” also includes substituted aryls that comprise aryl substituted on at least one member of the ring by, for example, C₁₋₄ alkyl, C₁₋₄ alkoxy, amino, hydroxy, phenyl, nitro, halo, carboxyalkyl or alkanoyloxy. Aryl groups include, for example, both substituted and unsubstituted phenyl, naphthyl, anthryl, biphenyl, methoxyphenyl, halophenyl, and the like.

As used herein the term “heteroaryl” refers to unsaturated aromatic carbocyclic radicals having a single ring, such as phenyl, or multiple condensed rings, such as naphthyl or anthryl with at least one non-carbon atom within at least one ring structure. The term “heteroaryl” also includes substituted heteroaryls, which are substituted at at least one or more positions by for example, C₁₋₄ alkyl, C₁₋₄ alkoxy, amino, hydroxy, phenyl, nitro, halo, carboxyalkyl or alkanoyloxy. Examples of heteroaryls include but are not limited to, piperidyl, pyrrolidinyl, pyrrolyl, morpholinyl, indolyl, indazolyl, benzimidazolyl, isoindolinyl, carbazolyl, or tetrahydroisoquinozinyl.

As used herein the term “alkyl” includes straight or branched saturated aliphatic hydrocarbon radicals, such as, for example, methyl, ethyl, propyl, isopropyl (1-methylethyl),

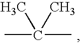

butyl, tert-butyl (1,1-dimethylethyl), and the like.

In one embodiment of the invention, X is —ZnBr. In another embodiment of the invention, A is phenyl. In yet another embodiment of the invention, Y is —CH₂. In a further embodiment of the invention, Z is piperidyl, pyrrolidinyl, pyrrolyl, morpholinyl, indolyl, indazolyl, benzimidazolyl, isoindolinyl, carbazolyl, or tetrahydroisoquinozinyl.

Specific examples of compounds of the invention include, but are not limited to, (4-(1-Piperidinylmethyl)phenyl) zinc bromide, 3-(1-Piperidinylmethyl)phenyl) zinc bromide, (2-(1-Piperidinylmethyl)phenyl) zinc bromide, (4-(1-Pyrrolindinylmethyl) phenyl) zinc bromide, (3-(1-Pyrrolidinylmethyl)phenyl) zinc bromide, (2-(1-Pyrrodinylmethyl)phenyl) zinc bromide, (4-(4-Morpholinylmethyl)phenyl) zinc bromide, (3-(4-Morpholinylmethyl)phenyl) zinc bromide, and (2-(4-Morpholinylmethyl)phenyl) zinc bromide.

Synthesis

Compounds of the invention can be synthesized using a method of the invention. A method of synthesizing compounds of the invention is illustrated below.

YW—A—V+H—Z→V—A—Y—Z

where W=Br,or Cl;

V=Cl, Br, or I; and

A, Y, and Z are as defined above

V—A—Y—Z+Zn→X—A—Y—Z

where A, X, V, Y, and Z, are as defined above

In the second step of this method of synthesizing the compounds of the invention, reactive zinc is utilized. In one embodiment, the reactive zinc is Rieke® Zinc (Rieke Metals, Inc., Lincoln Nebr.).

Different compounds of the invention can be synthesized by beginning with the specific halogen containing compound, YW—A—V and reacting one molar equivalent of it with 3 molar equivalents of Rieke® zinc for example. Generally, the mixture is refluxed for about 12 hours, depending on the specific aryl compound (YW—A—V) that is utilized. The reaction can be monitored, using known methods, such as for example, gas chromatography, to determine when the reaction has gone to completion.

One example of another embodiment of a method of producing halogen substituted amine compounds of the invention includes the following:

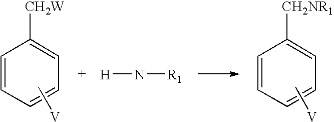

where R₁=aryl or heteroaryl;

and W and V are as defined above

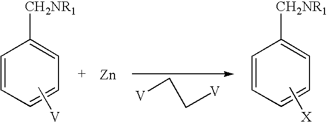

where X, V, R₁, and M are as defined above

In the second step of this method of synthesizing the compounds of the invention, reactive zinc is utilized. In one embodiment, the reactive zinc is Rieke® Zinc (Rieke Metals, Inc., Lincoln Nebr.).

Combinatorial Libraries

In one embodiment of the invention, compounds of the invention are utilized to produce combinatorial libraries. In one embodiment of the method of the invention, at least one compound of the invention can be immobilized onto a solid support, and various subsequent chemical steps could be undertaken to create libraries of compounds. In yet another embodiment, at least one compound not of the invention can be immobilized onto a solid support, and one or more compounds of the invention can be contacted with the immobilized compound as one of the steps in creating a combinatorial library. In a further embodiment of the invention, one compound of the invention is utilized as a beginning point for the production of a combinatorial library. In yet another embodiment, more than one compound of the invention is utilized as beginning points for the production of a combinatorial library.

One of skill in the art would know, given the disclosure of the specification, how to utilize one or more compounds of the invention in the production of a combinatorial library.

The production of combinatorial libraries can also include a number of other methods including, but not limited to, the use of scavenger resins, polymer-bound reagents, catch and release methods, and resin capture. Such strategies allow organic synthesis using simple, parallel product purification by filtration, avoiding silica-gel chromatography and/or extraction. New soluble polymer-supported catalysts, reagents and synthetic targets as adjuncts to solution-phase library development have also been utilized. Examples of some of these techniques include but are not limited to, covalent scavenger reagents to remove unwanted starting materials or by-products, resin capture reagents to transfer products in solution to solid phase for further modification, or “fishing-out” reagents to remove products from a complex reaction mixture. New resin supports for solid-phase chemistry developed by cross-linking strategies can also be utilized in combinatorial libraries made using at least one compound of the invention. Various methods of creating spacers between the molecules in the library and the solid support can also be utilized.

Intermediates

Compounds of the invention can also be utilized as intermediates in the synthesis of desired compounds. One of skill in the art could, utilizing knowledge generally available to those of skill in the art, formulate a synthesis for a desired compound that contains as a portion thereof, a compound or portion of a compound of the invention.

Compounds of the invention can be utilized as intermediates in reactions transferring at least one carbon and its electrons. The compounds can be used for a number of synthetic methods, including, but not limited to the production of alkanes through reaction with water (or other acids that are stronger acids than the organometallic reagent), and the production of alkanes through reaction with alkyl halides, and the production of alcohols through reaction with aldehydes, ketones, esters, or oxiranes.

This invention will be further characterized by the following examples. These examples are not meant to limit the scope of the invention, which has been fully set forth in the foregoing description. Variations within the scope of the invention will be apparent to those skilled in the art.

### EXAMPLES

Reagents used in the following Examples can be purchased from Aldrich Chemical Company (Milwaukee, Wis. or Allentown, Pa.), Fisher Scientific (Pittsburgh, Pa.), or RMI (Rieke Metals, Inc., Lincoln Nebr.).

### Example 1

### Synthesis of (4-(4-Morpholinylmethyl)phenyl) zinc bromide

2.2 moles of 4-bromobenzyliodide (Aldrich) was reacted with 423.4 g/450 mL (4.86 moles) morpholine (Aldrich) in 2 liters toluene (Fisher) at room temperature for about 1 day. The solution was rotovapped for about 12 hours to remove the solvent and leave the (4-(4- Morpholinylmethyl)phenyl) bromide.

The atmosphere of an oven-dried 250 mL 2-neck round bottom flask with condenser was filled with argon gas. 1.5 equivalents of Rieke® zinc in tetrahydrofuran (THF) (Rieke Metals, Inc.) was added via a plastic cannula (54 mL). 1 equivalent of the (4-(4- Morpholinylmethyl)phenyl) bromide was dissolved in 25 Ml of THF and then added via a cannula to the round bottom flask over 15 minutes. The reaction was heated to reflux for 4 hours. After 4 hours of reflux, the reaction had proceeded to completion. The progress of the reaction was followed by gas chromatography.

### Example 2

### Synthesis of (3-(4-Morpholinylmethyl)phenyl) zinc bromide

Three equivalents of Rieke® zinc (10 grams in 100 mL THF) was added to an oven dried 100 mL 2-neck round bottom flask with condenser, with an atmosphere of argon gas, with a plastic cannula. One equivalent of (3-(4- Morpholinylmethyl)phenyl) bromide was added neat via syringe over one minute. The reaction was heated to reflux for approximately 12 hours, to yield (3-(4-Morpholinylmethyl)phenyl) zinc bromide.

Claims:

1. A compound of formula I:
X—A—Y—Z (I)
wherein X is —ZnCl, —ZnBr, or —ZnI;
A is an aryl, or heteroaryl;
Y is C₁-C₆ alkyl; and
Z is a N containing heteroaryl that is bonded by the N.

2. A compound according to claim 1, wherein X is —ZnBr.

3. A compound according to claim 1, wherein A is phenyl.

4. A compound according to claim 1, wherein Y is CH₂.

5. A compound according to claim 1, wherein Z is piperidyl, pyrrolidinyl, pyrrolyl, morpholinyl, indolyl, indazolyl, benzimidazolyl, isoindolinyl, carbazolyl, or tetrahydroisoquinozinyl.

6. A combinatorial library formed using at least one compound according to claim 1.

7. A combinatorial library formed using more than one compound according to claim 1.
